(19) United States
(12) Patent Application Publication
(10) Pub. No.: US 2022/0125656 A1
(43) Pub. Date: Apr. 28, 2022

(54) METHODS AND APPARATUS FOR CELL AND GENE THERAPY AT POINT-OF-CARE LOCATION

(71) Applicant: APPLIED MATERIALS, INC., Santa Clara, CA (US)

(72) Inventors: Samer BANNA, San Jose, CA (US); Mukhles SOWWAN, Cupertino, CA (US); Mendy ERAD, Santa Clara, CA (US)

(21) Appl. No.: 17/496,242

(22) Filed: Oct. 7, 2021

Related U.S. Application Data
(60) Provisional application No. 63/104,149, filed on Oct. 22, 2020.

(51) Int. Cl. A61G 10/00; G16H 10/40; G16H 40/63; A61G 10/02; B01L 1/04

(52) U.S. Cl. CPC A61G 10/005; G16H 10/40; G16H 20/10; A61G 10/023; B01L 1/04; G16H 40/63

(57) ABSTRACT

Methods and apparatus for producing a treatment dose of engineered cells for individual patients at the point-of-care location. In some embodiments, a patient treatment system includes a mobile cell and gene processing station that provides individualized patient treatments at point-of-care locations. The cell and gene processing station may include a patient chamber configured to isolate a patient from an external environment, a first environment controller to adjust environmental parameters of the patient chamber, a processing chamber configured to isolate processing of patient cells from the external environment, a second environment controller to adjust environmental parameters of the processing chamber, a first apparatus configured to receive at least one blood sample from the patient, a second apparatus configured to process the blood sample, and a third apparatus configured to perform cell and gene engineering on the blood sample to create a treatment dose for the patient.

### CROSS-REFERENCE TO RELATED APPLICATIONS

This application claims benefit of U.S. provisional patent application Ser. No. 63/104,149, filed Oct. 22, 2020 which is herein incorporated by reference in its entirety.

### FIELD

Embodiments of the present principles generally relate to point-of-care treatment for cell and gene therapy.

### BACKGROUND

More doctors are looking to cell and gene therapy to thwart many diseases and genetic disorders. However, cell and gene therapy require an extremely sterile environment in which to process samples taken from the patient. In general, large expensive clean rooms must be built to house the equipment necessary to perform the cell and gene therapy processing. Because of the large expense in creating and maintaining the facilities, the centers for processing are mainly centrally located, requiring doctors and hospitals to send samples to the central location which may be in another city, state, or in some cases, a different country, delaying treatment while the samples are in transit, both to and from the facilities.

Accordingly, the inventors have provided methods and apparatus for processing cell and gene therapy treatments at the point-of-care location, dramatically reducing the cost of processing the samples and the delays in treatment due to remote processing of samples.

### SUMMARY

Methods and apparatus for cell and gene therapy at the point-of-care location are provided herein.

In some embodiments, a system for treating a patient may comprise a cell and gene processing station, wherein the cell and gene processing station is configured to be mobile and configured to provide individualized patient treatments at point-of-care locations, the cell and gene processing station may include at least one patient chamber that is configured to isolate a patient from an external environment, at least one first environment controller configured to adjust environmental parameters of the at least one patient chamber, a processing chamber that is configured to isolate processing of patient cells from the external environment, at least one second environment controller configured to adjust environmental parameters of the processing chamber, a first apparatus located within the processing chamber that is configured to receive at least one blood sample from the patient, a second apparatus located within the processing chamber that is configured to process the at least one blood sample from the patient, and a third apparatus located within the processing chamber that is configured to perform cell and gene engineering on the at least one blood sample from the patient to create a treatment dose for the patient.

In some embodiments, the system may further include wherein the second apparatus includes a cleaning apparatus that cleanses the second apparatus of byproducts related to the at least one blood sample, wherein the second apparatus includes a storage apparatus that is configured to preserve at least a portion of the at least one blood sample for a period of time of less than 24 hours and is configured to preserve at least a portion of the at least one blood sample for a period of time greater than 24 hours, wherein the first apparatus is configured to accept at least one blood sample directly from the patient via a direct connection to the patient, wherein the first apparatus further includes a communication apparatus located in proximity of the patient that is configured to relay information directly to the patient, wherein the communication apparatus is a speaker or a display, wherein the patient chamber and the processing chamber are negatively pressurized, wherein the third apparatus is configured to administer the treatment dose directly to the patient via a direct connection to the patient, wherein the third apparatus includes a first cubicle apparatus that is configured to hold blood cells for processing and a second cubicle apparatus that is configured to hold the first cubicle apparatus internally, and/or wherein the second cubicle apparatus is configured with sensors to monitor the blood cells or is configured with emitters to augment the processing of the blood cells.

In some embodiments, an apparatus for liquid handling for treatment processing may comprise a cell and gene handling apparatus configured for processing blood cells of a patient to produce a treatment dose of engineered cells for the patient, the cell and gene engineering apparatus may include a first cubicle apparatus configured with a plurality of tube-like liquid containers and a second cubicle apparatus configured with a plurality of sensors or emitters, wherein the first cubicle apparatus is configured to be placed wholly into the second cubicle apparatus, and wherein the plurality of sensors or emitters are configured to allow monitoring or influencing of a liquid in one or more of the plurality of tube-like liquid containers to facilitate in producing the treatment dose of engineered cells, wherein the first cubicle apparatus has a removable lid that is gastight when placed on the first cubicle apparatus, wherein the plurality of tube-like liquid containers are mounted to a bottom of the first cubicle apparatus and wherein the first cubicle apparatus is configured with access ports on the bottom to allow for dispensing of processed liquid from the plurality of tube-like liquid containers, wherein the second cubicle apparatus is configured to aid in producing of the treatment does by controlling the plurality of sensors or emitters to perform processing specific tasks, and/or wherein the second cubicle apparatus is configured with at least one supporting member on at least one side that have at least one sensor or emitter and wherein the at least one supporting member moves the at least one sensor or emitter vertically or horizontally across a side on a plurality of tracks by an actuator.

In some embodiments, a method of treating a patient may comprise receiving a first set of physical parameters from a patient medical data apparatus configured for an individual patient at a point-of-care location, receiving blood cells from the individual patient at the point-of-care location, applying a cell manufacturing process specific to the individual patient based, at least in part, on the first set of physical parameters from the patient medical data apparatus at the point-of-care location, engineering cells using the cell manufacturing process to create a treatment dose for the individual patient at the point-of-care location, and monitoring the individual patient using the patient medical data apparatus after administering the treatment dose to the individual patient at the point-of-care location.

In some embodiments, the method may further comprise receiving a second set of physical parameters from the patient medical data apparatus after applying the cell manufacturing process specific to the individual patient and altering the cell manufacturing process based, at least in part, on the second set of physical parameters from the patient medical data apparatus, and/or controlling care of the individual patient based, at least in part, on the cell manufacturing process via the patient medical data apparatus during the cell manufacturing process.

In some embodiments, a method of treating a patient may comprise selecting or customizing a medical treatment protocol for an individual patient at a point-of-care location, altering a patient medical data apparatus based on the medical treatment protocol at the point-of-care location, altering a cell engineering apparatus based on the medical treatment protocol at the point-of-care location, monitoring a progress of a cell manufacturing process and altering the cell manufacturing process based on the progress or deviations from the medical treatment protocol at the point-of-care location, producing a treatment dose of engineered cells from the cell manufacturing process for the individual patient at the point-of-care location, monitor effects of the treatment dose after administering the treatment dose of engineered cells to the individual patient using the patient medical data apparatus at the point-of-care location, and manufacturing additional or different treatment doses based on an advancement or deviation from the medical treatment protocol or success of the medical treatment protocol on the individual patient at the point-of-care location.

In some embodiments, the method may further include performing the method in parallel for a plurality of individual patients at the point-of-care location.

Other and further embodiments are disclosed below.

### BRIEF DESCRIPTION OF THE DRAWINGS

Embodiments of the present principles, briefly summarized above and discussed in greater detail below, can be understood by reference to the illustrative embodiments of the principles depicted in the appended drawings. However, the appended drawings illustrate only typical embodiments of the principles and are thus not to be considered limiting of scope, for the principles may admit to other equally effective embodiments.

FIG. 1 depicts a cross-sectional view of a CGT medicine manufacturing system in accordance with some embodiments of the present principles.

FIG. 2 depicts a cross-sectional view of a CMU in accordance with some embodiments of the present principles.

FIG. 3 depicts a cross-sectional view of a GEU in accordance with some embodiments of the present principles.

FIG. 4 depicts an isometric view of a cubicle that may be used as part of a GEU in accordance with some embodiments of the present principles.

FIG. 5 depicts an isometric view of a cubicle with injectors positioned to inject fluid into cell processing containers in accordance with some embodiments of the present principles.

FIG. 6 depicts an isometric view of a first cubicle fitting inside of a second cubicle in accordance with some embodiments of the present principles.

FIG. 7 depicts an isometric view of a cubicle with arrays of movable sensors in accordance with some embodiments of the present principles.

FIG. 8 depicts an isometric view of a cubicle that contains a cylinder with a plurality of capillary tubes in accordance with some embodiments of the present principles.

FIG. 9 depicts an isometric view of a cubicle sensor that may be interspersed between cell processing tubes in accordance with some embodiments of the present principles.

FIG. 10 is a method of using a CGT medicine manufacturing system in accordance with some embodiments of the present principles.

FIG. 11 is a method of using a CGT medicine manufacturing system in accordance with some embodiments of the present principles.

FIG. 12 is a method of using a CGT medicine manufacturing system in accordance with some embodiments of the present principles.

FIG. 13 is a method of using a CGT medicine manufacturing system in accordance with some embodiments of the present principles.

FIG. 14 depicts a plan view and a cross-sectional top down view of a mobile testing facility in accordance with some embodiments of the present principles.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

The methods and apparatus enable point-of-care location on-site manufacturing of medications for personalized cell and/or gene therapy (“CGT”). Various embodiments of the methods and apparatus enable per-person full process personally-configurable CGT medicines (treatment) and methods to use such apparatus to constantly alter and modify the manufacturing process in consideration of the medical condition of the treated person and physical response to the CGT medication and/or other medical treatment the patient may receive. In some embodiments, systems and apparatus enable on-demand CGT medication manufacturing with the flexibility to change the manufacturing process, including changes to the techniques, materials, monitoring and verification, and/or other manufacturing parameters—to enable real-time responses to real-time changes of the medical conditions of the patient. In some embodiments, an interactive, modular, adaptive CGT medicine manufacturing system includes the ability to receive medical data and/or physiological measured parameters gathered by a monitoring device carried by the user and, based on such medical data and parameters, may alter or change the CGT manufacturing process.

The examples found herein are for the sake of brevity and are not meant to limit the methods and apparatus of the present principles. The term “CGT medicine” refers to the medication (treatment) produced by “tailored” biological-engineering processes which are aimed to alter human cells and/or genes to address a specific medical condition or disease of a specific person. The term “Patient Medical Data” or “PMD” refers to medical data and/or measured physiological data, either in-vivo or not, of the patient that the CGT medicine is being manufactured for. The term “PMD apparatus” shall refer to a monitoring and/or measuring apparatus, capable of periodically measuring physiological parameters of a human, including, but not limited to, blood pressure, body temperature, ambient temperature, ambient humidity, blood chemistry, pulse, movement, oxidation, dehydration, skin color change, skin moisture change, medication levels in the body, toxicity levels of medications in the body, glucose, etc. The apparatus may include in-vivo measurements and/or skin-attached sensor measurements, and communications (wireless, wired, etc.). The PMD apparatus may also include medication administration so that medication can be given to the patient (either in-vivo or by other methods such as pills). The term “point-of-care” refers to the location where the patient is being treated or cared for and the like. The point-of-care may be the location where the patient has cell or gene samples taken and/or the location where the patient is administered a cell or gene treatment and the like.

Personalized CGT medicine manufacturing stands practically in direct contrast to the way medications and drugs have been developed for centuries. While traditional pharmaceutical companies have been working on finding and developing a medication that can fit as many people as possible—personalized CGT medicine places a specific individual as the sole end-client of the developed medication, which is tailored to the patient's specific medical needs and physical conditions. Personalizing CGT medicine presents a tremendous challenge—mainly in developing the proper operational model as CGT medicine manufacturing currently is very costly and requires advanced labs, skilled personal, specific materials, isolation conditions, is time consuming, and many other factors that can influence the successful outcome of the manufacturing process, and as a result—the successful use of the produced medication. Another influencing factor lies in the fact that most CGT manufacturing today is done in central facilities and not in the point-of-care (PoC) area close to the patient (e.g. in the clinics, hospitals, local research facilities, etc.). The remote processing is due, in part, to the lack of know-how, expertise, and facilities in the PoC area, partly due to the high cost of such systems that require large-enough targeted potential patients to justify local facility, regulatory consideration, and partly due to an intentional business model that enables vendors to charge premium top-dollar costs. The result is the same—the end-user of the potential medicine (who in most cases is the provider of the living cells that are used for tailoring the genetical-engineering medication process to the user's needs) is located far from the manufacturing site, and the logistical issues of how to safely and quickly provide the medication is added to the already-complex manufacturing process through the so called “cold-supply chain.”

Some companies have begun developing robotic PoC systems to enable localized CGT medicine manufacturing by trying to integrate conventional techniques based on reducing the scale of large stand-alone machines and systems. Some of the integrated conventional techniques are packaged as an “all-in-one” solution while some have single-use technologies based on existing concepts. The conventional techniques are based on miniaturization of the same off-site CGT medicine manufacturing facilities, while trying to replace human involvement with robots where possible and to enhance automation. However, the miniaturization of conventional techniques only serves to add another level of complexity to what is already a highly complex process. Each patient who is undergoing a CGT medicine process is unique—age, gender, genetics, prior-physical conditions, other background diseases and parameters, different nutrition, different effects of the treated disease on the patient's body, different progress of the disease, previous treatments the patient got before (for example, radiation, chemotherapy, surgery, etc.) that have influenced the patient's body, and even on-going treatments may influence the patient during the time spent to manufacture the CGT tailored medicine for the patient.

One must remember that many of the chemotherapy medicines and other types of medicines applied for diseases such as cancer, are toxic in nature (at some level), and have a severe effect on the human body, and the body organs, beyond medicine's immediate goal of attacking the cancer. The side effects must be taken into consideration when designing a tailored CGT medicine, as well when the patient starts being treated by the medicine. For example, the patient could be regarded as an “installation site” for a “treatment system” which is the CGT medicine. The process may take weeks, even if successful. The problem is that the “installation site” condition may significantly differ at the end of the period from the time the process had begun. Given that such medication is apriori targeted at already-sick people, the patient's physiological condition may not be the same as when the medication is ready for administering, thus, requiring the process to be started over again. Therefore, allowing flexibility in designing the treatment per the PoC judgment and based on the patient conditions are critical. One skilled in the art will understand that the methods and apparatus of the present principles may be applied to other applications than the examples given below.

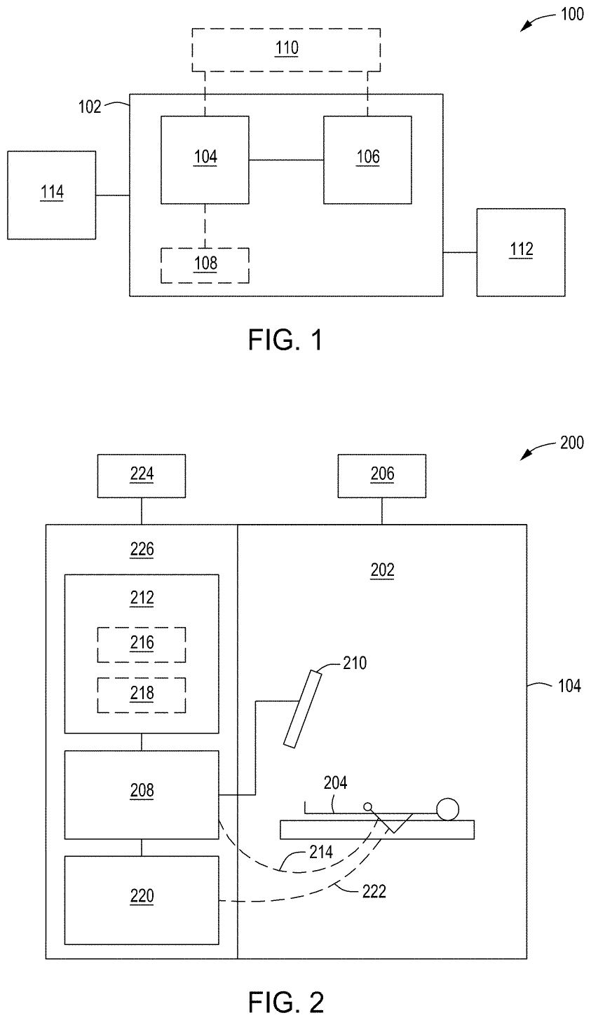

FIG. 1 depicts a cross-sectional view 100 of a CGT medicine manufacturing system 102 in accordance with some embodiments. The CGT medicine manufacturing system 102 includes at least one CGT manufacturing unit (CMU) 104 and at least one PMD apparatus 106. The CMU 104 and the PMD apparatus 106 may be fluidly coupled to share fluids and/or may also be electrically coupled via wired and/or wireless communications to share data. In some embodiments, the CMU 104 and the PMD apparatus 106 may be remote from each other. In some embodiments, a remote server 110 such as, but not limited to, a web server and the like may provide communication between the CMU 104 and the PMD apparatus 106. The communication via the remote server 110 may also include encryption to prevent disclosure of private information. The use of a remote server 110 allows for patient data to be obtained by the PMD apparatus 106 during all phases of the patient's treatment (e.g., during blood collection, during processing, and/or during and after administering of the treatment and the like). The PMD apparatus 106 may also be used to facilitate in controlling aspects of the patient's physiology directly and/or indirectly prior to administration of the treatment.

In some embodiments, the CGT medicine manufacturing system 102 may include a user interface 112 with the capability to choose a medical treatment protocol to apply to the manufacturing processes. The user interface 112 may enable a physician to customize a medical treatment process per patient, considering parameters such as, but not limited to, the data gathered from a PMD apparatus 106 and required CGT medicine, the changing physical condition of the patient, historical patient data, the type of disease, the disease's progress, and/or advancement and effects on the patient's body, and the like. The user interface 112 may also enable a physician to customize and/or change the CGT medicine manufacturing process and/or type. The user interface 112 may communicate with the CGT medicine manufacturing system 102 via a wired and/or wireless connection and/or via the remote server 110. In some embodiments, a control and monitoring unit 114 may be utilized to provide for data collection from various sensors and also to provide control over the processes used in the cell and gene therapy. In some embodiments, the control and monitoring unit 114 may incorporate machine learning to facilitate in controlling of the processing. The machine learning may utilize sensor/monitor data, current patient data, and/or historical patient data, at least in part, to determine which process to use and/or to determine changes to current processes being run in the CGT medicine manufacturing system 102. Changes may also be relayed to the patient via a PMD apparatus.

In some embodiments, the CMU 104 may include an actuator 108 that is configured to spin in any direction any of the units of the CMU 104, separately or in unison, with 2D and/or 3D movement, with configurable speeds and durations. FIG. 2 depicts a cross-sectional view 200 of the CMU 104 in accordance with some embodiments. In some embodiments, the CMU 104 is isolated in a chamber 226 to protect the processing from external contaminants. The environment of the chamber 226 may be negatively pressurized to ensure that any virus or other pathogens involved in the processing are kept isolated during the processing. In some embodiments, the chamber 226 may be enveloped by a positive pressure environment. The positive and negative pressures prevent foreign bodies (viruses, contaminants, etc.) from coming into the chamber 226 while keeping anything from inside the chamber 226 from leaving the chamber 226. An environmental controller 224 is used to control such parameters as, but not limited to, humidity, temperature, pressure, lighting, and other factors that may hinder or aid the processing, ensuring high quality treatments. In some embodiments, the environmental controller 224 may include an air purifier, a temperature controller, a pressure controller, and/or an air quality monitoring/alarm apparatus, and the like. The air quality monitoring/alarm apparatus may include temperature threshold alarms, pressure threshold alarms, and/or also include a pathogen detector and the like to notify care givers of harmful environmental conditions.

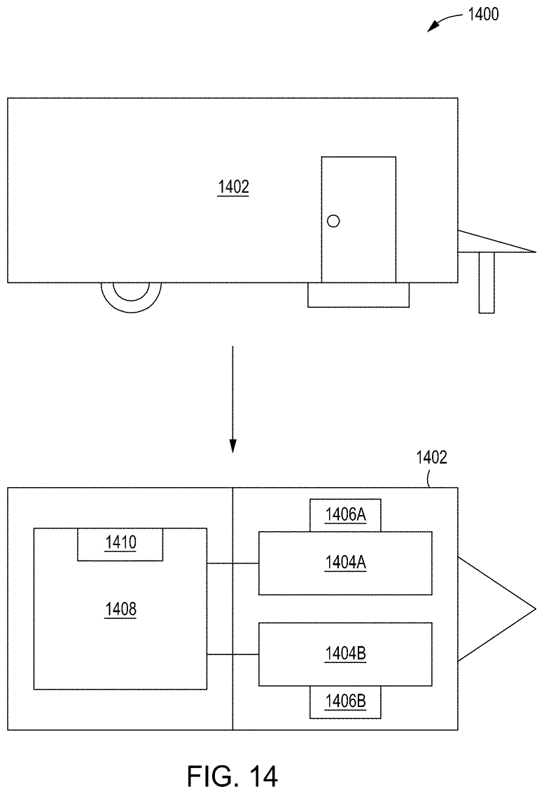

In some embodiments, the CMU 104 includes at least one patient chamber 202 that is configured to have at least one patient 204 inside. In some embodiments, the patient chamber 202 may have a negative pressure environment that is enveloped by a positive pressure environment. The positive and negative pressures prevent foreign bodies (viruses, contaminants, etc.) from coming into the patient chamber 202 while keeping anything from inside the patient chamber 202 from leaving the patient chamber 202. The patient chamber 202 may be used to take blood samples from the patient and/or to administer CGT medicine to the patient 204 in such conditions that ensure that the air in the patient chamber 202 is isolated from the air outside the patient chamber 202. The patient chamber 202 may also include at least one patient environment controller 206. In some embodiments, the patient environment controller 206 may include an air purifier, a temperature controller, a pressure controller, and/or an air quality monitoring/alarm apparatus, and the like. The air quality monitoring/alarm apparatus may include temperature threshold alarms, pressure threshold alarms, and/or also include a pathogen detector and the like to notify care givers of harmful environmental conditions. In some embodiments, the patient chamber 202 may reside external to the CMU 104 (see FIG. 14) and still be configured to allow interaction (fluid and/or electrical communications) between the CMU 104 and the patient chamber 202. In some embodiments, portions of the CMU 104 may operate internally and externally of the patient chamber 202 while maintaining interaction (fluid and/or electrical communications).

FIG. 14 depicts a plan and cross-sectional top down view 1400 of a mobile testing facility 1402 in accordance with some embodiments. The location of the mobile testing facility 1402 becomes the point-of-care location where the patient is treated. Being mobile, the point-of-care location may be brought to a locality near the patient for ease of patient and/or physician access. The mobile testing facility 1402 may include, but is not limited to, a trailer and the like that is mobile and may be brought to a hospital, physician's office, or patient location and the like for cell and gene therapy sessions. In some embodiments, the mobile testing facility 1402 may include a first patient chamber 1404A with a first patient environment controller 1406A and a second patient chamber 1404B with a second patient environment controller 1406B. Separate entrances or doors may be used on each side of the mobile testing facility 1402 to ensure isolation and/or patient privacy. Some embodiments may have a single patient chamber and environment controller or more than two patient chambers and patient environment controllers. Each patient's environment can be tailored specifically for that patient. The first patient chamber 1404A and the second patient chamber 1404B may be in fluid and/or electrical communication with a chamber 1408 used for processing the cell and gene therapy (CMU 104). The chamber 1408 includes an environmental controller 1410. In some embodiments, a PMD apparatus (not shown) may be provided for each patient to aid in gathering data for specific cell and gene therapy processing. The PMD apparatus may be a wired or a wireless connection to the chamber 1408.

Turning back to FIG. 2, the CMU 104 may also include at least one blood sampling and collection unit 208. The blood sampling and collection unit 208 may be manually operated, remotely operated by a remote operator, and/or automatically operated (e.g., but not limited to, robotic operation and the like). In some cases, the blood sampling and collection unit 208 may include an optional connection 214 directly to the patient to allow collection of samples over a period of time or at specific time intervals as required for collection and/or evaluation of the patient. In some embodiments, the blood sampling and collection unit 208 may have a patient communication apparatus 210 such as, but not limited to, a display and/or a speaker that allows the blood sampling and collection unit 208 to communicate instructions to the patient and/or results of the testing and the like. The CMU 104 may also include at least one blood processing unit 212. In some embodiments, the blood processing unit 212 may include apparatus to secure the blood drawn from the patient including apparatus to split the blood drawn into a plurality of storage units. The blood processing unit 212 preserves the blood by providing isolation of the blood from any external environment. In some embodiments, the blood processing unit 212 may further include blood storage 216 that allows the storing of parts of the drawn blood in different conditions based on different usages. For example, but not limited to, long storage conditions (e.g., storage for greater than 24 hours) for blood to be used as a backup supply, short storage conditions (e.g., storage for 24 hours or less) for blood to be used as a first treated sample, etc. In some embodiments, the blood processing unit 212 may also include a blood processing unit cleaner 218 that is configured to clean the blood processing unit 212 without any harmful effects to the blood within the blood processing unit 212.

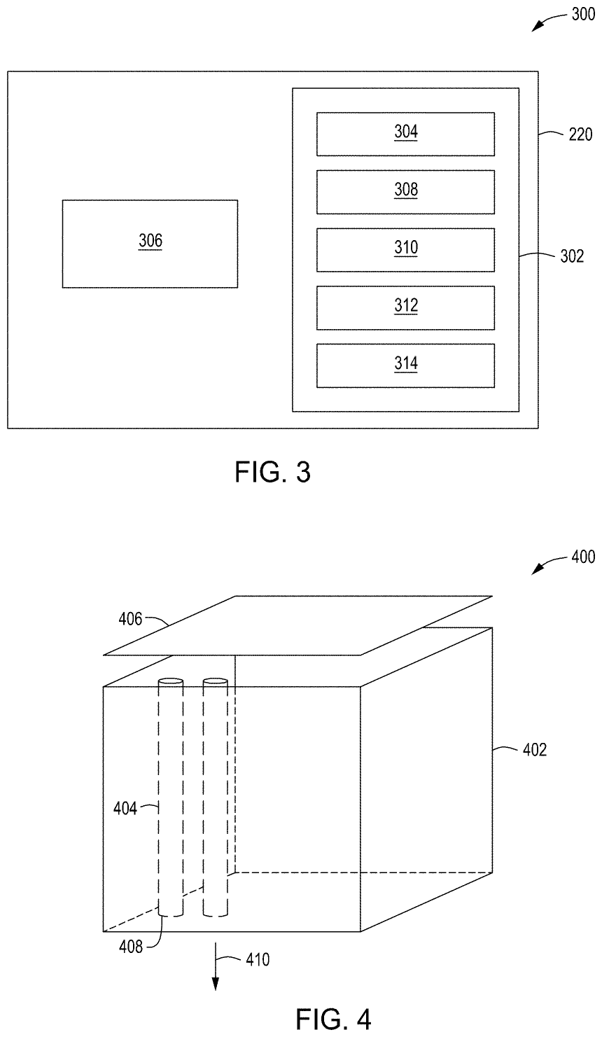

The CMU 104 may also include at least one genetic engineering unit (GEU) 220. The GEU 220 includes apparatus to extract cells and/or genes from the patient's blood and genetically engineer the cells and/or genes. In some embodiments, the GEU 220 may also interact with the patient to administer treatment 222. In some embodiments, the GEU 220 may be a replaceable unit that can be swapped out in order to process specific types of cells and/or genes. In some embodiments, a plurality of GEUs may be used to genetically engineer in parallel different types of cells. As depicted in the cross-sectional view 300 of FIG. 3, in some embodiments, the GEU 220 may include supporting apparatus 302 to cultivate, to provide nutrition, to monitor, to separate, and/or to safely dispose of treated cells. A monitoring apparatus 304 may include at least one camera and/or an alternative imaging system. The monitoring apparatus 304 may also include other types of sensors such as, but not limited to, light or color sensors, electrochemistry sensors, nano bead sensors, etc. The monitoring apparatus 304 may be configured to monitor, but not limited to, the vitality of the cells, the number and viability of the cells, the cultivation progress of the cells, the presence of contaminating materials, and/or the movement of treated cells with a fluidic tubing system 306 and the like. In some embodiments, the monitoring apparatus 304 may also include emitters to influence the manufacturing of the cells such as, but not limited to, light emitters, magnetic field emitters, and other emitters that impact the cells during processing.

In some embodiments, the GEU 220 may also include the fluidic tubing system 306 to channel cells between treating wells according to an applied procedure. The fluidic tubing system 306 may include the capability to cleanse the tubes in between processes to ensure that no contamination of the process occurs. In some embodiments, the GEU 220 may also include a detection apparatus 308 with a capability to detect and alert to the presence of a contamination in any part of the GEU 220. In some embodiments, the contamination may include chemicals and/or any material other than the treated cells which is not part of the materials used as part of the genetic engineering process. In some embodiments, the contamination may include chemicals and/or any material other than the treated cells which are part of the materials used as part of the genetic engineering process but are in presence in a quantity larger than a pre-defined threshold of the applied procedure and/or procedure's stage of processing.

In some embodiments, the GEU 220 may include at least one environmental apparatus 310 for controlling temperature, humidity, and/or light. The environmental apparatus 310 may be different for each part of the GEU 220. The environmental apparatus 310 may be controlled to change the applied settings per type of cells being treated and/or treatment stage. The environmental apparatus 310 may also be controlled to change the applied settings per cell nutrition process, per cell cultivation process to enable better control of the process, and/or to expedite process time and/or to maximize manufactured cells. In some embodiments, the GEU 220 may have at least one processor and/or memory 312 that is configured to perform specific routines for the processing and genetic engineering of cells. In some embodiments, the GEU 220 may have a capability to calculate the yield at each stage of the genetic manufacturing process. In some embodiments, the GEU 220 may have the capability to enable parallel manufacturing processes for the same end-result of CGT medicine from the same type of cells and may include a parallel processing apparatus 314 to monitor, analyze, and compare the yield and/or quantity at each stage per process and at the end of the manufacturing process. In some embodiments, the GEU 220 may be enabled to operate per process so processes may be added, repeated, and/or removed as needed or as specific patient parameters change.

In some embodiments, units of the CMU 104 may be configured to be disposable and/or reusable. Reusable units may also be configured to have apparatus for cleansing and purifying of the unit. The units of the CMU 104 may be of any size and shape such as, but not limited to, a cube, an octagon, and/or a cylinder and the like. In some embodiments, a GEU 220 may be in the form of one or more cubicles with an inner hollow cavity to enable insertion of a monitoring apparatus and the like. In some embodiments, the cubicles fit one inside of the other, with one cubicle being consumable and used for holding tube-like liquid containers while the other permanent outer cube contains controls, monitors, and/or emitters. The cubicles may be formed of glass and/or any other transparent and bio compatible material and the like. One of the cubicles may also include a fluidic tubing apparatus and/or cell wells and the like. The cubicle material may be shaped to enable better optical monitoring of the fluidic tubing apparatus and/or cell wells. For example, a cell well may be encompassed by a glass that also functions as a magnifying lens. Or, for example, the glass may be shaped to enable better detection of a specific light frequency. The monitoring of an inner unit may also be configured to rotate so different kinds of monitoring elements may be directed to different areas of the cubicle. In some embodiments, a GEU 220 may be in a form of a first cubicle that is placed within a second cubicle that is configured with a monitoring apparatus that monitors from all sides of the first cubicle (all 6 sides).

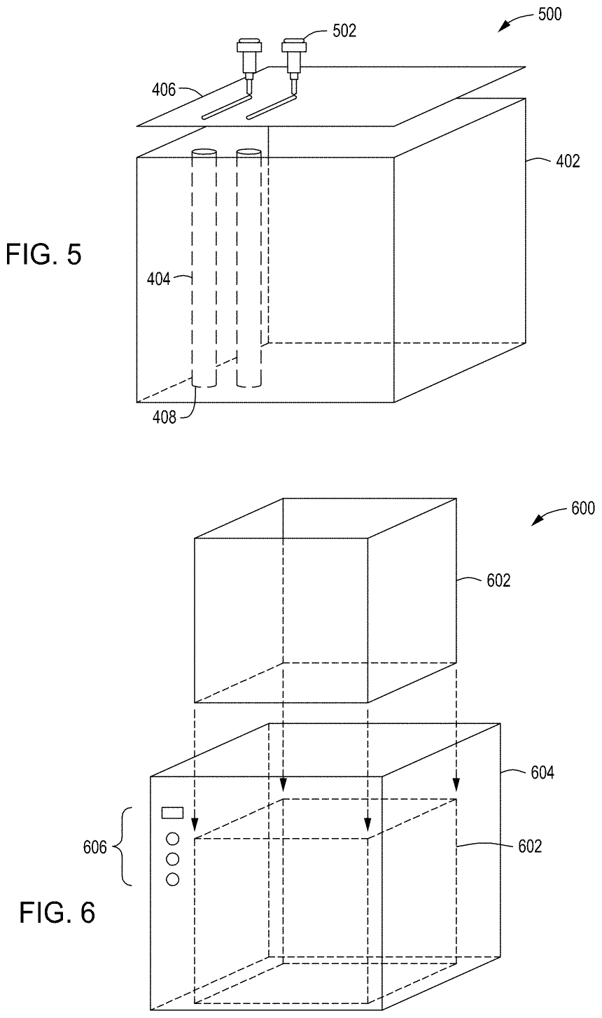

FIG. 4 depicts an isometric view 400 of an inner cubicle 402 that may be used as part of a GEU in accordance with some embodiments. The inner cubicle 402 is a six-sided cube that houses at least one cell processing container 404 (e.g., a tube-like liquid container, etc.). In some embodiments, the cell processing container 404 has a tubular shape, but the shape may be of any form. The inner cubicle 402 has a lid 406 that is removable to allow access to the cell processing container 404 to aid in filling the cell processing container 404 or removing contents from the cell processing container 404. The lid 406 is gas tight sealed when seated on the inner cubicle 402 and is configured to withstand forces from a centrifuge and configured to prevent entry of air particles. In some embodiments, portions of a bottom 408 of the cell processing container 404 may be opened to allow an engineered cell treatment solution (treatment dose) to be drained or removed 410 from the cell processing container 404 at the end of a process. In some embodiments, the cell processing containers 404 may be individually drained as each cell processing container 404 may contain a different engineered cell treatment or cell treatments at different stages within the process. FIG. 5 depicts an isometric view 500 of the inner cubicle 402 with injectors 502 positioned to inject fluid into the cell processing containers in accordance with some embodiments. The injectors 502 may be of a manual type controlled by a technician or physician and/or may be of an automatic type that is controlled by the GEU during a process.

FIG. 6 depicts an isometric view 600 of a first cubicle 602 fitting inside of a second cubicle 604 in accordance with some embodiments. In some embodiments, the second cubicle 604 may be outfitted with an array of sensors, emitters, and controls 606 that allow for monitoring and processing of the contents of the first cubicle 602. In some embodiments, the first cubicle 602 is used to hold tubes or capillaries filled with cell samples from a patient that are to be processed. When the first cubicle 602 is placed inside the second cubicle 604, the second cubicle 604 allows for six-sided access to the first cubicle 602 for monitoring and emitting apparatus. In some embodiments, the second cubicle 604 is a permanent component and the first cubicle 602 is a disposable or consumable component that is less costly to manufacture due to the absence of sensors and/or controls. In some embodiments, the second cubicle 604 may have cameras, lasers at different wavelengths, lighting, heating, cooling, magnetic fields, and/or electronic memory to store collected data and the like from one or more of the six sides of the first cubicle 602.

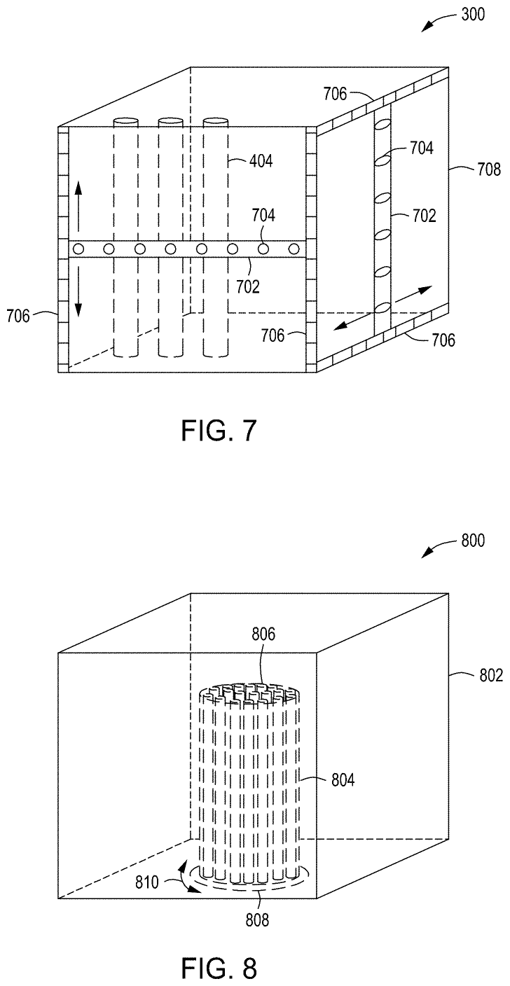

FIG. 7 depicts an isometric view 700 of an cubicle 708 with arrays of sensors/emitters 704 in accordance with some embodiments. The cubicle 708 is an outer cubicle. An inner cubicle that contains the cell processing containers 404 is not shown for better clarity. Movable supporting members 702 are used to hold sensors/emitters 704 in position. The movable supporting members 702 may be configured to move along rails or tracks 706 so that the cell processing containers 404 may be monitored from top-to-bottom and side-to-side or any other angle. Actuators such as, but not limited to, stepper motors and the like can be used to control the movement of the movable supporting members 702 to obtain desired data. The sensors/emitters 704 may include different types of sensors and/or emitters (e.g., cameras, lasers, magnets, etc.) to aid in identification, processing, and/or measurement of various parameters and the like during processing. In FIG. 7, only two sides are shown with tracks 706 and movable supporting members 702 with sensors/emitters 704, but all six sides or less of the cubicle 708 may have tracks 706 and movable supporting members 702 with sensors/emitters 704.

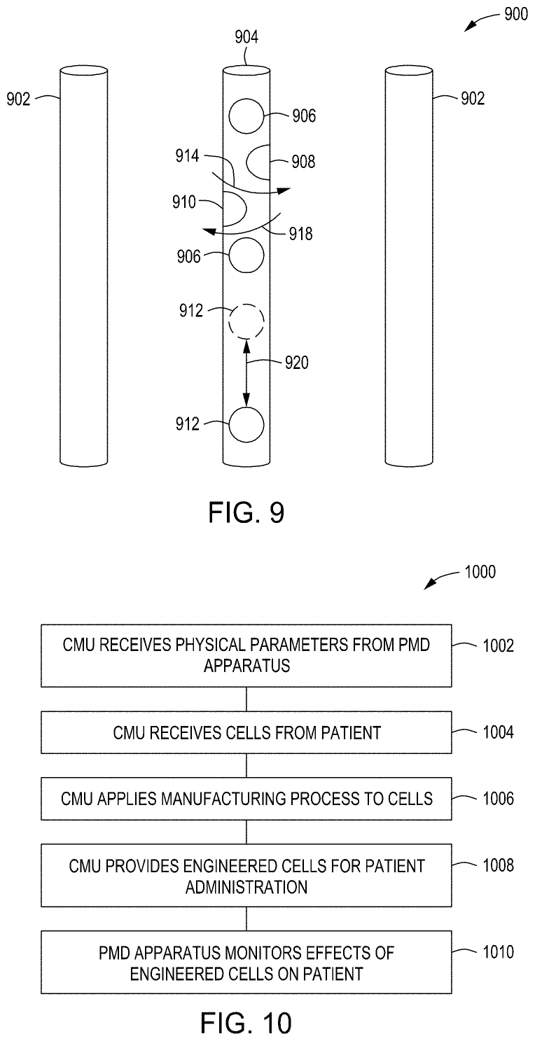

FIG. 8 depicts an isometric view 800 of a cubicle 802 that contains a cylinder 804 with a plurality of capillary tubes 806 in accordance with some embodiments. The capillary tube may allow for tens or hundreds of tests to be performed in one processing session. The cylinder 804 rests on a rotatable plate 808 that may be rotated clockwise and/or counterclockwise direction 810 to expose the capillary tubes 806 to a multitude of sensors during processing. FIG. 9 depicts an isometric view 900 of a cubicle sensor/emitter 904 that may be interspersed between cell processing tubes 902 in accordance with some embodiments. The cubicle sensor/emitter 904 is disposed one or more cell processing tubes in order to obtain data or to influence the process from many different angles. The cubicle sensor/emitter 904 may have one or more stationary sensors and/or emitters 906, one or more rotating sensors and/or emitters 908, 910, and one or more linearly moving sensors or emitters 912. In some embodiments, the rotating sensors and/or emitters may rotate in a counterclockwise direction 914 and/or a clockwise direction 918. In some embodiments, the linearly moving sensors/emitters may move in an up and/or down direction 920. A cubicle may have one or more of the cubicle sensors/emitters. In some embodiments, the cubicle sensors/emitters may reside in a first cubicle and a second cubicle with provisions for the sensors/emitters to protrude through the bottom of the second cubicle may be placed into the first cubicle for processing.

FIG. 10 is a method 1000 of using a CGT medicine manufacturing system in accordance with some embodiments. In block 1002, a CMU receives physical parameters from a PMD apparatus. In some embodiments, the PMD apparatus is applied to a patient for self-monitoring prior to the beginning of a CGT medicine manufacturing process in the CMU. For example, the patient may be required by a physician to monitor the patient's physical parameters for two months before cells are taken from the patient for processing in the CMU. In block 1004, the CMU receives the cells taken from the patient at the appropriate time. In block 1006, the CMU applies a manufacturing process to the cells to engineer cells for the patient's treatment. In some embodiments, a physician may select the manufacturing process and/or the manufacturing process may be selected based on patient information and the like. In block 1008, the CMU completes the engineering of the cells for patient treatment and the cells are administered to the patient. In block 1010, a PMD apparatus is used to monitor the effects of the engineered cells on the patient. In some embodiments, a physician may then use the information obtained by the PMD apparatus to make changes in subsequent manufacturing processes.

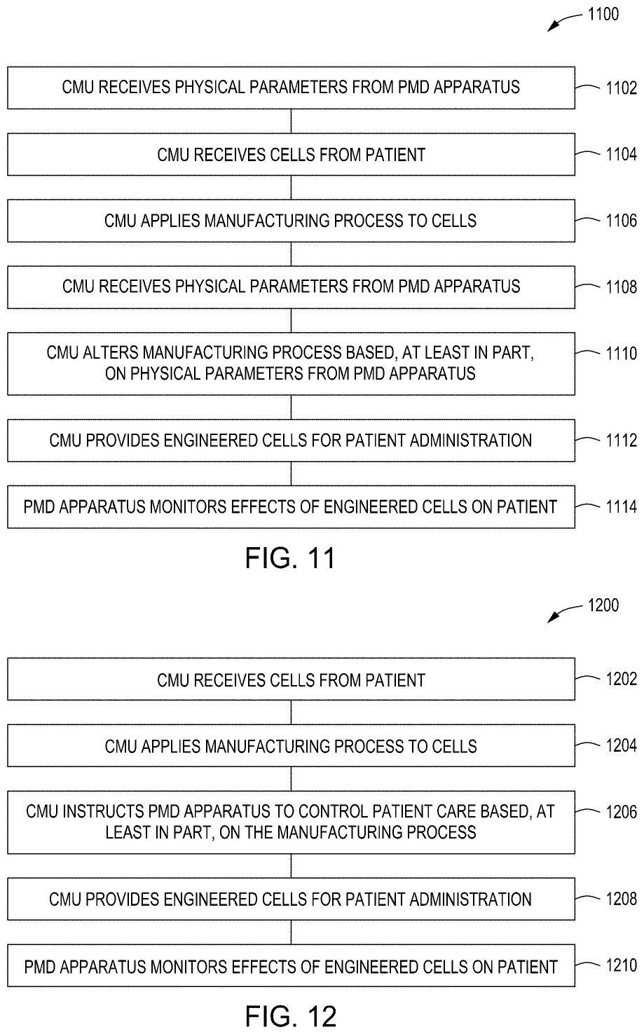

FIG. 11 is a method 1100 of using a CGT medicine manufacturing system in accordance with some embodiments. In block 1102, a CMU receives physical parameters from a PMD apparatus. In some embodiments, the PMD apparatus is applied to a patient for self-monitoring prior to the beginning of a CGT medicine manufacturing process in the CMU. For example, the patient may be required by a physician to monitor the patient's physical parameters for two months before cells are taken from the patient for processing in the CMU. In block 1104, the CMU receives the cells taken from the patient at the appropriate time. In block 1106, the CMU applies a manufacturing process to the cells to engineer cells for the patient's treatment. In some embodiments, a physician may select the manufacturing process and/or the manufacturing process may be selected based on patient information and the like. In block 1108, the CMU receives physical parameters with regard to a patient from a PMD apparatus that the patient is using to continue to monitor the patient's physical parameters while the manufacturing process is ongoing. In block 1110, the CMU alters the ongoing manufacturing process based, at least in part, on the physical parameters from the PMD apparatus that is monitoring the patient under treatment. By altering the manufacturing process based on the current condition of the patient, the engineered cells will be produced for maximum effectiveness based on the patient's current condition, rather than a static condition taken days, weeks, or even months prior to cell processing. In block 1112, the CMU completes the engineering of the cells for patient treatment, and the cells are administered to the patient. In block 1114, a PMD apparatus is used to monitor the effects of the engineered cells on the patient. In some embodiments, a physician may then use the information obtained by the PMD apparatus to make changes in subsequent manufacturing processes.

FIG. 12 is a method 1200 of using a CGT medicine manufacturing system in accordance with some embodiments. In block 1202, a CMU receives the cells taken from the patient at the appropriate time. In block 1204, the CMU applies a manufacturing process to the cells to engineer cells for the patient's treatment. In some embodiments, a physician may select the manufacturing process and/or the manufacturing process may be selected based on patient information and the like. In block 1206, the CMU instructs a PMD apparatus applied to a patient to control patient care based, at least in part, on the manufacturing process. The manufacturing process may be reliant on certain physical parameters of the patient remaining constant or changing to a different level when the engineered cells are to be administered. The CMU increases the effectiveness of the engineered cells by controlling the patient's physical condition through the PMD apparatus. In block 1208, the CMU completes the engineering of the cells for patient treatment and the cells are administered to the patient. In block 1210, the PMD apparatus is used to monitor the effects of the engineered cells on the patient. In some embodiments, a physician and/or a deep learning program may then use the information obtained by the PMD apparatus to make changes in subsequent manufacturing processes.

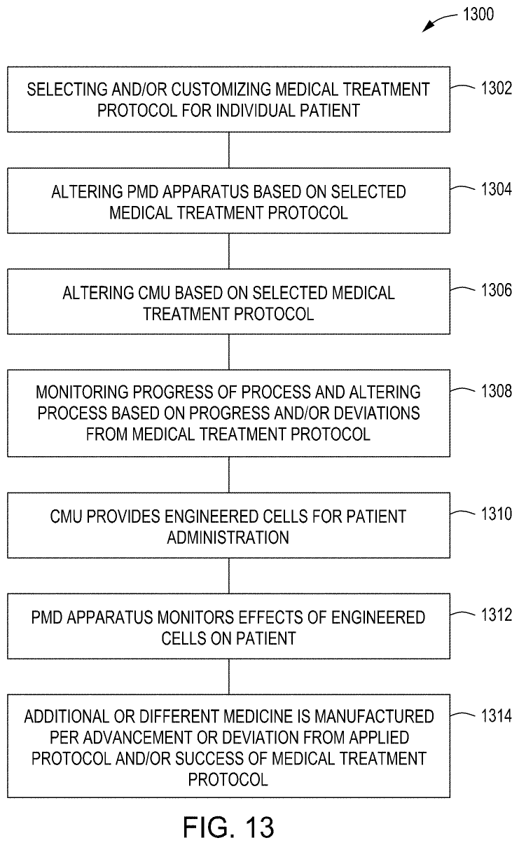

FIG. 13 is a method 1300 of using a CGT medicine manufacturing system in accordance with some embodiments. In block 1302, a medical treatment protocol is selected and/or customized for an individual patient. In some embodiments, the selection and/or customization may be made by a physician for a patient and/or the CMU may automatically determine the selection and/or customization based on the patient's physical parameters. In block 1304, a PMD apparatus applied to a patient is altered based on the selected medical treatment protocol. In some embodiments, the PMD apparatus may be altered by a physician for a patient and/or the CMU may automatically alter the PMD apparatus. In block 1306, the CMU is altered based on the selected medical treatment protocol. In some embodiments, the CMU may be altered manually or the CMU may automatically be altered based upon the selected medical treatment protocol. In block 1308, the progress of the process is monitored and the process is altered based on the progress and/or deviations from the medical treatment protocol. In some embodiments, the monitoring may be performed by a physician and/or by the CMU and/or the PMD apparatus which may automatically update the process accordingly. In block 1310, the CMU completes the engineering of the cells for patient treatment and the cells are administered to the patient. In block 1312, the PMD apparatus is used to monitor the effects of the engineered cells on the patient. In some embodiments, a physician and/or a deep learning program may then use the information obtained by the PMD apparatus to make changes in subsequent manufacturing processes. In block 1314, additional or different medicine is manufactured per advancement or deviation from the applied medical treatment protocol and/or success of the medical treatment protocol.

Embodiments in accordance with the present principles may be implemented in hardware, firmware, software, or any combination thereof. Embodiments may also be implemented as instructions stored using one or more computer readable media, which may be read and executed by one or more processors. A computer readable medium may include any mechanism for storing or transmitting information in a form readable by a machine (e.g., a computing platform or a “virtual machine” running on one or more computing platforms). For example, a computer readable medium may include any suitable form of volatile or non-volatile memory. In some embodiments, the computer readable media may include a non-transitory computer readable medium.

While the foregoing is directed to embodiments of the present principles, other and further embodiments of the principles may be devised without departing from the basic scope thereof.

1. A system for treating a patient, comprising:
a cell and gene processing station, wherein the cell and gene processing station is configured to be mobile and configured to provide individualized patient treatments at point-of-care locations, the cell and gene processing station includes:
at least one patient chamber that is configured to isolate a patient from an external environment;
at least one first environment controller configured to adjust environmental parameters of the at least one patient chamber;
a processing chamber that is configured to isolate processing of patient cells from the external environment;
at least one second environment controller configured to adjust environmental parameters of the processing chamber;
a first apparatus located within the processing chamber that is configured to receive at least one blood sample from the patient;
a second apparatus located within the processing chamber that is configured to process the at least one blood sample from the patient; and
a third apparatus located within the processing chamber that is configured to perform cell and gene engineering on the at least one blood sample from the patient to create a treatment dose for the patient.

2. The system of claim 1, wherein the second apparatus includes a cleaning apparatus that cleanses the second apparatus of byproducts related to the at least one blood sample.

3. The system of claim 1, wherein the second apparatus includes a storage apparatus that is configured to preserve at least a portion of the at least one blood sample for a period of time of less than 24 hours and is configured to preserve at least a portion of the at least one blood sample for a period of time greater than 24 hours.

4. The system of claim 1, wherein the first apparatus is configured to accept at least one blood sample directly from the patient via a direct connection to the patient.

5. The system of claim 1, wherein the first apparatus further includes a communication apparatus located in proximity of the patient that is configured to relay information directly to the patient.

6. The system of claim 5, wherein the communication apparatus is a speaker or a display.

7. The system of claim 1, wherein the patient chamber and the processing chamber are negatively pressurized.

8. The system of claim 1, wherein the third apparatus is configured to administer the treatment dose directly to the patient via a direct connection to the patient.

9. The system of claim 1, wherein the third apparatus includes a first cubicle apparatus that is configured to hold blood cells for processing and a second cubicle apparatus that is configured to hold the first cubicle apparatus internally.

10. The system of claim 9, wherein the second cubicle apparatus is configured with sensors to monitor the blood cells or is configured with emitters to augment the processing of the blood cells.

11. An apparatus for liquid handling for treatment processing, comprising:
a cell and gene handling apparatus configured for processing blood cells of a patient to produce a treatment dose of engineered cells for the patient, the cell and gene engineering apparatus includes:
a first cubicle apparatus configured with a plurality of tube-like liquid containers; and
a second cubicle apparatus configured with a plurality of sensors or emitters,
wherein the first cubicle apparatus is configured to be placed wholly into the second cubicle apparatus, and wherein the plurality of sensors or emitters are configured to allow monitoring or influencing of a liquid in one or more of the plurality of tube-like liquid containers to facilitate in producing the treatment dose of engineered cells.

12. The apparatus of claim 11, wherein the first cubicle apparatus has a removable lid that is gastight when placed on the first cubicle apparatus.

13. The apparatus of claim 11, wherein the plurality of tube-like liquid containers are mounted to a bottom of the first cubicle apparatus and wherein the first cubicle apparatus is configured with access ports on the bottom to allow for dispensing of processed liquid from the plurality of tube-like liquid containers.

14. The apparatus of claim 11, wherein the second cubicle apparatus is configured to aid in producing of the treatment does by controlling the plurality of sensors or emitters to perform processing specific tasks.

15. The apparatus of claim 11, wherein the second cubicle apparatus is configured with at least one supporting member on at least one side that have at least one sensor or emitter and wherein the at least one supporting member moves the at least one sensor or emitter vertically or horizontally across a side on a plurality of tracks by an actuator.

16. A method of treating a patient, comprising:
receiving a first set of physical parameters from a patient medical data apparatus configured for an individual patient at a point-of-care location;
receiving blood cells from the individual patient at the point-of-care location;
applying a cell manufacturing process specific to the individual patient based, at least in part, on the first set of physical parameters from the patient medical data apparatus at the point-of-care location;
engineering cells using the cell manufacturing process to create a treatment dose for the individual patient at the point-of-care location; and
monitoring the individual patient using the patient medical data apparatus after administering the treatment dose to the individual patient at the point-of-care location.

17. The method of claim 16, further comprising:
receiving a second set of physical parameters from the patient medical data apparatus after applying the cell manufacturing process specific to the individual patient; and
altering the cell manufacturing process based, at least in part, on the second set of physical parameters from the patient medical data apparatus.

18. The method of claim 16, further comprising:
controlling care of the individual patient based, at least in part, on the cell manufacturing process via the patient medical data apparatus during the cell manufacturing process.

19. The method of claim 16, further comprising:
selecting or customizing a medical treatment protocol for the individual patient at the point-of-care location;
altering the patient medical data apparatus based on the medical treatment protocol at the point-of-care location;
altering a cell engineering apparatus based on the medical treatment protocol at the point-of-care location;
manufacturing additional or different treatment doses based on an advancement or deviation from the medical treatment protocol or success of the medical treatment protocol on the individual patient at the point-of-care location.

20. Performing the method of claim 19 in parallel for a plurality of individual patients at the point-of-care location.
